(19) United States
(12) Patent Application Publication
(10) Pub. No.: US 2026/0325695 A1
(43) Pub. Date: Oct. 22, 2026

(54) HYDROGEN PEROXIDE VAPORIZATION SYSTEM

(71) Applicant: STERIS Corporation, Mentor, OH (US)

(72) Inventors: Michael A. Varcho, Mentor, OH (US); Nathan Pohl, Wickliffe, OH (US); Dale J. Kitchen, Chardon, OH (US); Albert Giebel, Mentor, OH (US); Elizabeth Simandl, Mentor, OH (US); Kent Denbow, Painesville, OH (US)

(21) Appl. No.: 19/642,980

(22) Filed: Apr. 9, 2026

Related U.S. Application Data
(60) Provisional application No. 63/790,203, filed on Apr. 17, 2025.

(51) Int. Cl. A61L 2/208; C01B 15/01; F24H 1/14

(52) U.S. Cl. CPC A61L 2/208; C01B 15/01; F24H 1/142; A61L 2202/11; A61L 2202/13; A61L 2202/15

(57) ABSTRACT

A hydrogen peroxide vaporization system may include a process path for decontamination and comprising a boiler subsystem, the boiler subsystem configured to heat and vaporize a solution comprising hydrogen peroxide, and optionally for conditioning and aerating a space comprising hydrogen peroxide. A hydrogen peroxide vaporization system may include one or more air moving devices configured for moving air about the solution comprising hydrogen peroxide.

### CROSS-REFERENCE TO REALTED APPLICATION

This patent application claims the benefit of U.S. Provisional Patent Application No. 63/790,203, filed April 17, 2025, which is incorporated by reference herein in its entirety.

### BACKGROUND

Vaporized hydrogen peroxide generators represent technique for biodecontamination. These systems utilize hydrogen peroxide vapor to achieve high-level biodecontamination at relatively low temperatures. This technology is useful in healthcare, pharmaceutical, and research environments where maintaining sterility is desirable, offering a versatile and reliable solution that combines effectiveness with compatibility for sensitive materials.

### SUMMARY

In some aspects, the techniques described herein relate to a hydrogen peroxide vaporization system including: a process path for decontamination and including a boiler subsystem, the boiler subsystem configured to heat and vaporize a solution including hydrogen peroxide, and optionally for conditioning and aerating a space including hydrogen peroxide; and one or more air moving devices configured for moving air about the solution including hydrogen peroxide.

In some aspects, the techniques described herein relate to a hydrogen peroxide vaporization system including: a first process path for decontamination and including a boiler subsystem, the boiler subsystem configured to heat and vaporize a solution including hydrogen peroxide; a second process path for conditioning and aerating a space including hydrogen peroxide and optionally including: a desiccant accessory, an air heater, and a catalyst; and one or more air moving devices configured for moving air about the solution including hydrogen peroxide.

In some aspects, the techniques described herein relate to a method of vaporizing hydrogen peroxide including: heating a solution including hydrogen peroxide from an ambient temperature to produce a non-condensing vapor; conditioning and aerating a space ; and facilitating air mixing within an application volume using an air moving device.

In some aspects, the techniques described herein relate to a method of vaporizing hydrogen peroxide including: flowing a solution including hydrogen peroxide through a double helical flow path; heating the solution including hydrogen peroxide from an ambient temperature to produce a non-condensing vapor; directing the vapor through a first process path for decontamination; directing air through a second process path optionally including: a desiccant accessory, an air heater, and a catalyst for conditioning and aerating; and facilitating air mixing within an application volume using axial blowers.

In some aspects, the techniques described herein relate to a boiler for vaporizing hydrogen peroxide including: a double helical flow path adapted to heat a hydrogen peroxide solution from ambient temperature; a heat transfer contact area sized for continuous flow rates in a range of from about 0.5 to about 15 grams per minute; a metal housing enclosing the double helical flow path; and wherein the boiler is configured to produce non-condensing vapor from a solution including about 30 wt% to about 70% hydrogen peroxide.

### BRIEF DESCRIPTION OF THE FIGURES

The drawings illustrate generally, by way of example, but not by way of limitation, various embodiments of the present disclosure.

FIG. 1 is a schematic diagram of a hydrogen peroxide vaporization system.

FIG. 2 is a schematic diagram of another hydrogen peroxide vaporization system.

FIG. 3 is a schematic diagram of a hydrogen peroxide vaporization system.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt% to about 5 wt% of the composition is the material, or about 0 wt% to about 1 wt%, or about 5 wt% or less, or less than or equal to about 4.5 wt%, 4, 3.5, 3, 2.5, 2, 1.5, 1, 0.9, 0.8, 0.7, 0.6, 0.5, 0.4, 0.3, 0.2, 0.1, 0.01, or about 0.001 wt% or less, or about 0 wt%.

Vaporized hydrogen peroxide (VHP) generators are an advanced solution for biodecontamination and can be useful in environments where traditional methods might be too harsh or impractical. These generators use hydrogen peroxide vapor to achieve high-level biodecontamination, making them suitable for many different applications such as for heat-sensitive materials. In general, a vaporized hydrogen peroxide process operates at low temperatures such as from about 15 ⁰C to about 40 ⁰C, about 20 ⁰C to about 30 ⁰C, less than, equal to, or greater than about 15 ⁰C, 16, 17, 18, 19, 20, 21, 22, 23, 24, 25, 26, 27, 28, 29, 30, 31, 32, 33, 34, 35, 36, 37, 38, 39, or about 40 ⁰C, allowing for a thorough decontamination without compromising the integrity of items such as medical devices, laboratory equipment, and electronic components.

In general, vaporized hydrogen peroxide generators are desirable for their ability to achieve a high level (e.g., a 6+ log) of reduction in microbial load, effectively eliminating about 99.9999% of microorganisms. This level of efficacy is desirable in healthcare settings, where maintaining sterility is desirable for patient safety. The dry process used in vaporized hydrogen peroxide biodecontamination avoids potential damage caused by moisture, making it suitable for a wide range of applications, including the biodecontamination of pre-filled syringes, implants, and other sensitive medical devices.

In addition to their effectiveness, vaporized hydrogen peroxide generators can be designed to meet global standards, ensuring that the biodecontamination process is reliable, repeatable, and safe. For example, vaporized hydrogen peroxide generators, such as those described herein can be equipped with advanced monitoring and control features, allowing users to track and verify each biodecontamination cycle. This level of control is desirable in regulated industries, where documentation and traceability are desirable if not required.

Overall, vaporized hydrogen peroxide generators offer a versatile and reliable solution for achieving high-level biodecontamination in various settings. Their ability to biodecontaminate sensitive materials without causing substantial damage to the materials, combined with increased compliance with various standards, makes them a valuable asset in healthcare, pharmaceutical, and research environments. Whether for routine biodecontamination of medical devices or laboratory equipment, vaporized hydrogen peroxide biodecontamination provides an effective and efficient method for ensuring sterility and safety.

In general, a vaporized hydrogen peroxide biodecontamination process begins with placing the items to be biodecontaminated inside an enclosed space. The space is then dehumidified and heated to remove any moisture, this is beneficial for the effectiveness of the hydrogen peroxide vapor in that it allows for higher concentrations of hydrogen peroxide to be added while staying below its condensation point. Next, liquid hydrogen peroxide is vaporized and then injected into the enclosed space. This creates a vapor that evenly spreads throughout the space. The items to be biodecontaminated are exposed to this vapor for a specified period, during which the vapor disrupts the cellular structures of microorganisms. After the exposure period, the chamber is aerated to remove any residual hydrogen peroxide vapor, ensuring the items are safe to handle and free from harmful residues. Once aeration is complete, the biodecontaminated items can be safely removed. This entire process is carefully monitored and controlled to ensure consistency and reliability, making vaporized hydrogen peroxide biodecontamination highly effective for heat and moisture-sensitive materials in healthcare, pharmaceutical, and research settings.

Vaporized hydrogen peroxide biodecontamination offers several advantages over traditional methods like ethylene oxide (EO) and steam biodecontamination. One of the primary benefits is it’s ability to operate at lower temperatures, making it suitable for heat-sensitive materials that might be damaged by high-temperature processes used in steam biodecontamination. This low-temperature operation ensures that delicate items, such as certain medical devices and electronic components, can be effectively biodecontaminated without compromising their integrity.

Another advantage of vaporized hydrogen peroxide biodecontamination is its shorter cycle times compared to traditional methods. Steam biodecontamination, for instance, often requires longer exposure periods and cooling times, while vaporized hydrogen peroxide can achieve effective biodecontamination more quickly. This efficiency can be particularly beneficial in high-demand settings where rapid turnaround is essential.

Vaporized hydrogen peroxide biodecontamination also tends to leave minimal residues, unlike ethylene oxide, which can leave toxic residues that require extensive aeration before the items are safe to use. Vaporized hydrogen peroxide can also be exhausted into the atmosphere where it can naturally decay. This makes vaporized hydrogen peroxide a safer and more environmentally friendly option. Additionally, vaporized hydrogen peroxide generators are designed to meet stringent global standards, ensuring reliable and repeatable biodecontamination cycles, which is crucial for maintaining consistent sterility in regulated industries.

Overall, vaporized hydrogen peroxide biodecontamination provides a versatile and efficient alternative to traditional methods, offering advantages in terms of temperature sensitivity, cycle time, and safety. This makes it a valuable option for a wide range of applications in healthcare, pharmaceutical, and research environments.

Vaporized hydrogen peroxide generators have a wide range of applications in healthcare due to their effectiveness and versatility. As an example, they can be used in the biodecontamination of medical devices and surgical instruments. Vaporized hydrogen peroxide biodecontamination can additionally be beneficial for items that are sensitive to heat and moisture, such as endoscopes, catheters, and other delicate equipment. This is because the low-temperature process ensures that these devices are thoroughly biodecontaminated without being damaged, maintaining their functionality and extending their lifespan.

In addition to medical devices, vaporized hydrogen peroxide generators can be used for room and surface decontamination in healthcare facilities. This includes operating rooms, patient rooms, and laboratories, where maintaining a sterile environment is needed to prevent infections and improve patient safety. The vapor can penetrate hard-to-reach areas, ensuring comprehensive decontamination of all surfaces.

Another example of a suitable application is in the biodecontamination of packaging materials and pharmaceutical products. Vaporized hydrogen peroxide biodecontamination is effective in ensuring that packaging is free from contaminants, which is essential for maintaining the sterility of the products inside. This is particularly useful, as an example, for items like pre-filled syringes and other single-use medical products.

Overall, vaporized hydrogen peroxide biodecontamination provides a reliable and efficient method for achieving high-level biodecontamination in various healthcare settings. Its ability to biodecontaminate sensitive materials without causing damage, combined with its effectiveness in decontaminating large areas, makes it an invaluable tool in maintaining sterility and safety in healthcare environments.

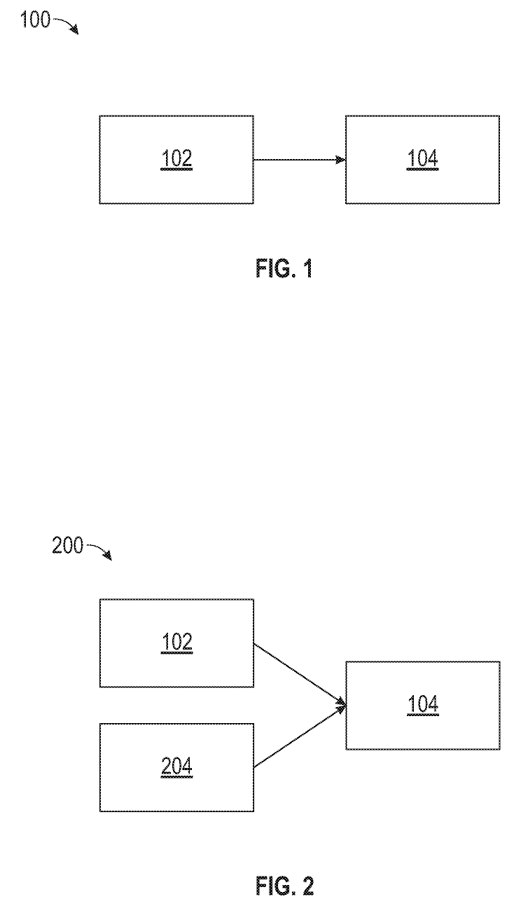

Disclosed herein is hydrogen peroxide vaporization system 100, which can be used for any of the aforementioned applications. FIG. 1 is a schematic representation of hydrogen peroxide vaporization system 100. Hydrogen peroxide vaporization 100 includes a single process path. The single process path includes boiler subsystem 102 and decontamination chamber 104.

Boiler subsystem 102 is configured to heat and vaporize a solution that includes hydrogen peroxide. As generally understood, vaporization of hydrogen peroxide is the process by which it transitions from its liquid form to a vapor state. This occurs when hydrogen peroxide (H₂O₂) is heated to its boiling point, which for a 100 wt% H₂O₂ solution, is about 150.2°C (302.4°F) at standard atmospheric pressure.

The solution including hydrogen peroxide includes about 30 wt% to about 70% hydrogen peroxide, about 35 wt% to about 60% hydrogen peroxide, less than, equal to, or greater than about 30 wt%, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or about 70 wt% hydrogen peroxide, all based on the liquid concentration of hydrogen peroxide. The balance of the solution including hydrogen peroxide is generally water.

Boiler system 102 produces a non-condensing vapor of hydrogen peroxide. A non-condensing vapor of hydrogen peroxide refers to hydrogen peroxide in its vapor state that does not condense into a liquid under typical conditions. This is achieved by vaporizing hydrogen peroxide in a controlled manner, ensuring that it remains as a vapor without forming liquid droplets.

Devices used to condition and aerate a hydrogen peroxide solution may include a stirring or agitation system, which can include a magnetic stirrer or a mechanical agitator, used to introduce air into the solution and help provide an even distribution of hydrogen peroxide.

To bring the hydrogen peroxide from ambient temperature to its boiling temperature, boiler subsystem 102 can include a double helical flow path configured to heat the hydrogen peroxide solution from ambient temperature. The double helical flow path of boiler subsystem 102, can generally include a helical profile about a heating cartridge.

A helical profile features a flow path wound in a spiral or helix shape around a heating cartridge. This design enhances the heater's surface area, improving heat transfer efficiency and allows for more uniform heating. The helical winding can help ensure that the heat is evenly distributed along the length of the flow path. This helps to allow for consistent and precise temperature control. The flow path can include a metal tube or sheath that can include, stainless-steel, aluminum, or other metals or alloys. Aluminum may be desirable for its flexibility. In some examples, the flow path can be a multi-component system. For example, the flow path can include metal sheath can include an aluminum sheath surrounded by a stainless-steel sheath. The metal sheath helps to provide durability and resistance to corrosion, while maximizing contact with the surrounding material, to provide efficient thermal conductivity.

The hydrogen peroxide solution is continuously flowed through the helical flow path around the cartridge heater. The flow of the hydrogen peroxide solution is accomplished through one or more air moving devices or liquid pumps. An example of a suitable air moving device can include an axial blower. An example of a liquid pump is a positive displacement pump. The hydrogen peroxide solution in liquid form is typically moved by the liquid pump and once it is injected as a vapor into an airstream, it can be moved by the air moving device.

The cartridge heater is sized for a continuous flow rate of the hydrogen peroxide solution in a range of from about 0.5 to about 15 grams per minute, about 1 to about 2 grams per minute, less than, equal to, or greater than about 0.5 grams per minute, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or about 10 grams per minute or any higher amount.

Sizing the cartridge heater includes several factors. The flow rate of the hydrogen peroxide solution directly impacts the amount of heat energy required to achieve the desired temperature. The helical profile of the heater exchanger or flow path allows for enhanced surface area contact, helping to achieve substantially efficient and uniform heat distribution. To determine the appropriate heater size, the power output is calculated based on the specific heat capacity of the hydrogen peroxide solution, the heat of vaporization of the hydrogen peroxide solution, the initial and target temperatures, and the flow rate. Additionally, the thermal conductivity of the surrounding material and the heater's insertion length is considered to ensure optimal heat transfer.

Hydrogen peroxide vaporization system 100 can include several accessories and/or components to aid in function. For example, hydrogen peroxide vaporization system 100 can include a desiccant accessory, an air heater, a catalyst, or a mixture thereof. A desiccant accessory is used to remove moisture from the air before it enters the vaporization chamber, to help ensure that the hydrogen peroxide vaporizes properly without condensation issues. For example, reusable desiccant cartridges can be regenerated and reused, which can make the process more environmentally friendly and cost-effective. Alternatively, the desiccant cartridge can be a single use cartridge. An air heater can be used to heat the incoming air, enhancing the vaporization rate of hydrogen peroxide and help provide a substantially consistent and uniform distribution of the vapor. This can be helpful in applications requiring precise control over the concentration and distribution of the vapor, such as in biodecontamination processes. A catalyst can be used to accelerate the decomposition of hydrogen peroxide into water and oxygen, which can provide the ability for safe re-entry of the space, or ready-to-use decontaminated products. As an example, catalysts like platinum or palladium can be used in these systems due to their good efficiency and reliability.

The hydrogen peroxide vapor generated can be used for relatively small volume decontamination. For example, the hydrogen peroxide vaporization system can be used to decontaminate solutions or an apparatus having a volume no larger than 5 cubic meters, in a range of from about 0.5 to about 4 cubic meters, about 1 to about 3 cubic meters, less than, equal to, or greater than about 0.5 cubic meters, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or about 5 cubic meters.

FIG. 2 is a schematic representation of hydrogen peroxide vaporization system 200. Hydrogen peroxide vaporization system 200 differs relative to system 100 in that system 200 includes second process path 204. Second process path 204 is used for conditioning and aerating the space about to be decontaminated by the hydrogen peroxide solution. System 100 only included elements for conditioning and aerating as optional components.

Second process path 204 is positioned in parallel with first process path 102 and upstream decontamination chamber 104. Second process path includes any of the desiccant accessory, the air heater, the catalyst, or combination thereof described herein above. Typically, only one of first process path 102 and second process path 204 will be active at any time.

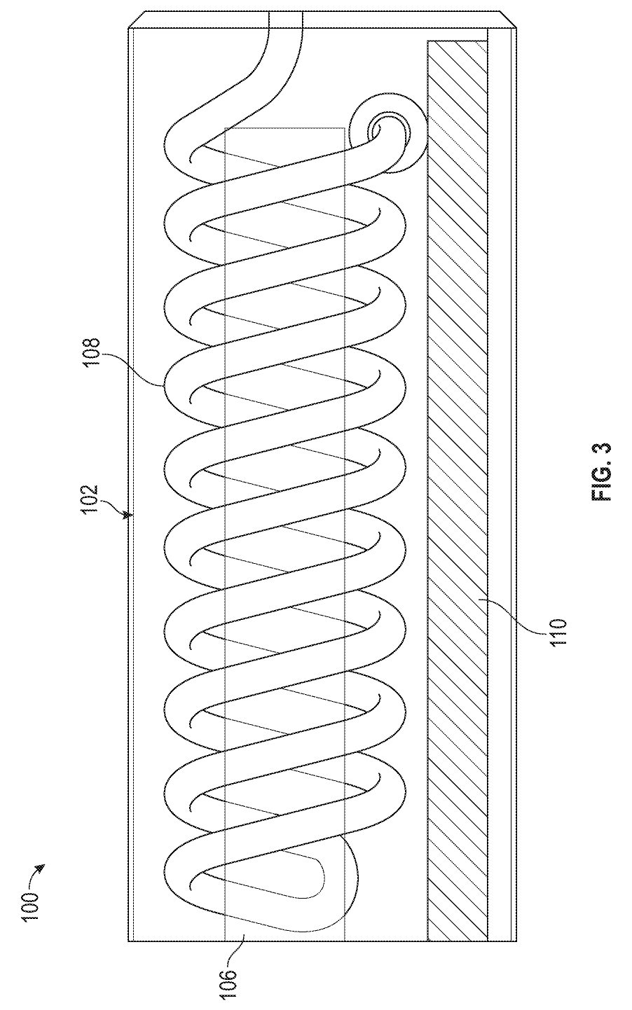

FIG. 3 is a schematic representation of hydrogen peroxide vaporization system 100. As shown, subsystem 102 includes cartridge heater 106 and flow path 108. As shown flow path 108 is wrapped in a double helical pattern about cartridge heater 106. FIG. 3 also shows sensor 110. Sensor 110 is a temperature sensing device that that can sense the temperature of system 100 along flow path 108. Sensor 110 can control when cartridge heater 106 turns on or off.

Boiler subsystem 102 can be made my subtractive or additive manufacturing. Additionally, any component or entire pathway of either system 100 or 200 can be formed through subtractive or additive manufacturing. With respect to additive manufacturing, one of a suitable method is direct metal laser sintering (DMLS), also referred to as selective laser melting (SLM). This process involves using a high-power laser to fuse layers of metallic powder into solid parts. The laser is guided by a computer-aided design (CAD) file, which allows for precise control over the geometry of the part being produced. DMLS is particularly advantageous for creating complex shapes and internal structures that would be difficult or impossible to achieve with traditional manufacturing methods.

Another additive manufacturing technique that can be employed is powder bed fusion (PBF). This method involves spreading a thin layer of powdered material over a build platform and then selectively fusing the powder using a laser or electron beam. PBF is highly versatile and can produce parts with fine details and high strength-to-weight ratios. This makes it suitable for manufacturing components that require both structural integrity and intricate designs, such as those found in hydrogen peroxide vaporization systems.

In some examples, post-processing steps may be used to enhance the properties of the additively manufactured parts. These steps can include heat treatment to improve mechanical properties, surface finishing to achieve the desired smoothness, and coating to provide additional protection against thermal and chemical stresses. For instance, thermal barrier coatings can be applied to parts that will be exposed to high temperatures, ensuring their longevity and performance in demanding environment.

### Examples

Various embodiments of the present disclosure can be better understood by reference to the following Examples which are offered by way of illustration. The present disclosure is not limited to the Examples given herein.

Aluminum Material of construction of the Boiler was chosen primarily due to Aluminum’s high thermal conductivity and availability. The double helical liquid flow path within the Boiler was designed utilizing aspects of existing 2 fluid spiral heat exchangers in industrial cooling applications that maximize the surface area contact between 2 fluids, 1 hot and 1 cold. The double helix allows for a more uniform and controlled increase in liquid temperature and minimizes the temperature gradient along the length of the Boiler. The cross-sectional diameter of the liquid flow path was designed to accommodate the low H₂O₂ flow rates while still maintaining “pipe flow” (where the liquid does not have a free surface and maintains full surface contact with its flow path). This maximizes heat transfer area to the liquid and allows higher energy efficiency in a smaller footprint. The length of the liquid flow path is then determined utilizing the circumference of the liquid cross-section, the energy required to vaporize the continuous flow of liquid, and the total heat transfer contact area between the liquid flow path and the Aluminum body. Direct Metal Laser Sintering was chosen as the primary means of manufacturing the Boiler subsystem due to the complexity of the double helical internal design that would otherwise require modification for conventional machining processes. In operation, the boiler’s temperature generated by the cartridge heater existing inside the middle of the helix, is controlled to 10 to 15 degrees C above the liquid’s boiling point to prevent partial vapor conversion and reduce H₂O₂ concentration destruction. This setpoint can be subject to change based on placement of temperature measurement device and temperature gradient of the boiler. Ambient temperature liquid H₂O₂ enters the inlet of the boiler at a continuous flow rate and is heated and vaporized before leaving the Boiler outlet where it is injected into an air stream to be delivered to the target area requiring decontamination. The primary benefit of the Boiler subsystem is its minimized footprint for use in a Decontamination System and its minimized energy consumption due to designed heating of solely the liquid process and only passive heating of the surrounding air process.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed may be resorted to by those of ordinary skill in the art, and that such modifications and variations are considered to be within the scope of embodiments of the present disclosure.

### Exemplary Aspects

The following exemplary aspects are provided, the numbering of which is not to be construed as designating levels of importance:

Aspect 1 provides a hydrogen peroxide vaporization system comprising:
a process path for decontamination and comprising a boiler subsystem, the boiler subsystem configured to heat and vaporize a solution comprising hydrogen peroxide, and optionally for conditioning and aerating a space comprising hydrogen peroxide; and
one or more air moving devices configured for moving air about the solution comprising hydrogen peroxide.

Aspect 2 provides the system of Aspect 1, further comprising a desiccant accessory, an air heater, a catalyst, or a combination thereof.

Aspect 3 provides the system of any of Aspects 1 or 2, wherein the one or more air moving devices comprise an axial blower.

Aspect 4 provides the system of any of Aspects 1-3, wherein the boiler subsystem comprises:
a double helical flow path configured to heat the hydrogen peroxide solution from ambient temperature; and
a heat transfer contact area sized for continuous flow rates.

Aspect 5 provides the system of Aspect 4, wherein the heat transfer contact area is sized for continuous flow rates in a range of from about 0.5 to about 15 grams per minute.

Aspect 6 provides the system of any of Aspects 4 or 5, wherein the heat transfer contact area is sized for continuous flow rates in a range of from about 1 to about 2 grams per minute.

Aspect 7 provides the system of any of Aspects 1-6, wherein:
the solution comprising hydrogen peroxide comprises about 30 wt% to about 70% hydrogen peroxide; and
the boiler subsystem produces non-condensing vapor.

Aspect 8 provides the system of Aspect 7, wherein the solution comprising hydrogen peroxide comprises about 35 wt% to about 60% hydrogen peroxide.

Aspect 9 provides the system of any of Aspects 1-8, wherein the system is configured to decontaminate solutions having a volume no larger than 5 cubic meters.

Aspect 10 provides the system of any of Aspects 1-9, wherein the system is configured to decontaminate solutions having a volume in a range of from about 0.5 to about 4 cubic meters.

Aspect 11 provides the system of any of Aspects 1-10, wherein the boiler subsystem comprises a flexible aluminum tubing wrapped in a helical geometry around a cartridge heater.

Aspect 12 provides the system of Aspect 11, wherein the aluminum tubing is disposed inside a stainless-steel sleeve.

Aspect 13 provides a hydrogen peroxide vaporization system comprising:
a first process path for decontamination and comprising a boiler subsystem, the boiler subsystem configured to heat and vaporize a solution comprising hydrogen peroxide;
a second process path for conditioning and aerating a space comprising hydrogen peroxide and optionally comprising:
a desiccant accessory,
an air heater, and
a catalyst; and
one or more air moving devices configured for moving air about the solution comprising hydrogen peroxide.

Aspect 14 provides the system of Aspect 13, wherein the boiler subsystem comprises:
a double helical flow path configured to heat the hydrogen peroxide solution from ambient temperature; and
a heat transfer contact area sized for continuous flow rates in a range of from about 0.5 to about 3 grams per minute.

Aspect 15 provides the system of Aspect 14, wherein the heat transfer contact area is sized for continuous flow rates in a range of from about 1 to about 2 grams per minute.

Aspect 16 provides the system of any of Aspects 13-15, wherein the one or more air moving devices comprise axial blowers.

Aspect 17 provides the system of any of Aspects 13-16, wherein:
the solution comprising hydrogen peroxide comprises about 30 wt% to about 70% hydrogen peroxide; and
the boiler subsystem produces non-condensing vapor.

Aspect 18 provides the system of any of Aspects 13-17, wherein the system is configured to decontaminate solutions having a volume in a no larger than 5 cubic meters.

Aspect 19 provides the system of Aspect 18, wherein the system is configured to decontaminate solutions having a volume in a range of from about 0.5 to about 4 cubic meters.

Aspect 20 provides the system of any of Aspects 13-19, wherein the boiler subsystem comprises a flexible aluminum tubing wrapped in a helical geometry around a cartridge heater.

Aspect 21 provides the system of Aspect 20, wherein the aluminum tubing is disposed inside a stainless-steel sleeve.

Aspect 22 provides the system of any of Aspects 13-21, wherein the boiler subsystem is fabricated using metal laser sintering.

Aspect 23 provides a method of vaporizing hydrogen peroxide comprising:
heating a solution comprising hydrogen peroxide from an ambient temperature to produce a non-condensing vapor;
conditioning and aerating a space comprising the hydrogen peroxide; and
facilitating air mixing within an application volume using an air moving device.

Aspect 24 provides the method of Aspect 23, wherein the air moving device comprises an axial blower.

Aspect 25 provides the method of any of Aspects 23 or 24, further comprising directing the vapor through a first process path for decontamination.

Aspect 26 provides the method of any of Aspects 23-25, further comprising directing air through a second process path optionally comprising:
a desiccant accessory,
an air heater, and
a catalyst for conditioning and aerating; and
facilitating air mixing within an application volume using axial blowers.

Aspect 27 provides the method of Aspect 26, further comprising flowing a solution comprising hydrogen peroxide through a double helical flow path.

Aspect 28 provides the method of Aspect 27, wherein the solution comprising hydrogen peroxide flows in a range of from about 0.5 to about 3 grams per minute.

Aspect 29 provides the method of any of Aspects 27 or 28, wherein the solution comprising hydrogen peroxide flows in a range of from about 1 to about 2 grams per minute.

Aspect 30 provides the method of any of Aspects 23-29, wherein the solution comprising hydrogen peroxide comprises about 30 wt% to about 70% hydrogen peroxide.

Aspect 31 provides the method of any of Aspects 23-30, wherein the solution comprising hydrogen peroxide comprises about 35 wt% to about 60% hydrogen peroxide.

Aspect 32 provides the method of any of Aspects 23-31, further comprising decontaminating a volume of the solution comprising hydrogen peroxide having a volume no larger than 5 cubic meters.

Aspect 33 provides the method of any of Aspects 23-32, further comprising decontaminating a volume of the solution comprising hydrogen peroxide having a volume in a range of from about 0.5 to about 4 cubic meters.

Aspect 34 provides the method of any of Aspects 27-33, wherein the double helical flow path is formed by wrapping thin-walled flexible aluminum tubing around a cartridge heater.

Aspect 35 provides a method of vaporizing hydrogen peroxide comprising:
flowing a solution comprising hydrogen peroxide through a double helical flow path;
heating the solution comprising hydrogen peroxide from an ambient temperature to produce a non-condensing vapor;
directing the vapor through a first process path for decontamination;
directing air through a second process path optionally comprising:
a desiccant accessory,
an air heater, and
a catalyst for conditioning and aerating; and
facilitating air mixing within an application volume using axial blowers.

Aspect 36 provides the method of Aspect 35, wherein the solution comprising hydrogen peroxide flows at a rate in a range of from about 0.5 to about 3 grams per minute.

Aspect 37 provides the method of any of Aspects 35 or 36, wherein the heat transfer contact area is sized for continuous flow rates in a range of from about 1 to about 2 grams per minute.

Aspect 38 provides the method of any of Aspects 35-37, wherein the solution comprising hydrogen peroxide comprises about 30 wt% to about 70% hydrogen peroxide.

Aspect 39 provides the method of any of Aspects 35-38, wherein the solution comprising hydrogen peroxide comprises about 35 wt% to about 60% hydrogen peroxide.

Aspect 40 provides the method of any of Aspects 35-39, further comprising decontaminating a volume of the solution comprising hydrogen peroxide having a volume no larger than 5 cubic meters

Aspect 41 provides the method of any of Aspects 35-40, further comprising decontaminating a volume of the solution comprising hydrogen peroxide having a volume in a range of from about 0.5 to about 4 cubic meters.

Aspect 42 provides the method of any of Aspects 35-41, wherein the double helical flow path is formed by wrapping thin-walled flexible aluminum tubing around a cartridge heater.

Aspect 43 provides a boiler for vaporizing hydrogen peroxide comprising:
a double helical flow path adapted to heat a hydrogen peroxide solution from ambient temperature;
a heat transfer contact area sized for continuous flow rates in a range of from about 0.5 to about 3 grams per minute;
a metal housing enclosing the double helical flow path; and
wherein the boiler is configured to produce non-condensing vapor from a solution comprising about 50 wt% to about 70% hydrogen peroxide.

Aspect 44 provides the boiler of Aspect 43, wherein the double helical flow path comprises thin-walled aluminum tubing wrapped around a cartridge heater.

Aspect 45 provides the boiler of any of Aspects 43 or 44, wherein the metal housing comprises a stainless-steel sleeve in contact with the wrapped tubing.

Aspect 46 provides the boiler of any of Aspects 43-45, wherein the boiler is fabricated using metal laser sintering.

Aspect 47 provides the boiler of any of Aspects 43-46, wherein the solution comprises about 57 wt% to about 62 wt% hydrogen peroxide.

What is claimed is:

1. A hydrogen peroxide vaporization system comprising:
a process path for decontamination and comprising a boiler subsystem, the boiler subsystem configured to heat and vaporize a solution comprising hydrogen peroxide, and optionally for conditioning and aerating the solution comprising hydrogen peroxide; and
one or more air moving devices configured for moving air about the solution comprising hydrogen peroxide.

2. The system of claim 1, further comprising a desiccant accessory, an air heater, a catalyst, or a combination thereof.

3. The system of claim 1, wherein the one or more air moving devices comprise an axial blower.

4. The system of claim 1, wherein the boiler subsystem comprises:
a double helical flow path configured to heat the hydrogen peroxide solution from ambient temperature; and
a heat transfer contact area sized for continuous flow rates.

5. The system of claim 4, wherein the heat transfer contact area is sized for continuous flow rates in a range of from about 0.5 to about 3 grams per minute.

6. The system of claim 1, wherein:
the solution comprising hydrogen peroxide comprises about 30 wt% to about 70% hydrogen peroxide; and
the boiler subsystem produces non-condensing vapor.

7. The system of claim 1, wherein the system is configured to decontaminate solutions having a volume no larger than 5 cubic meters.

8. The system of claim 1, wherein the boiler subsystem comprises a flexible aluminum tubing wrapped in a helical geometry around a cartridge heater.

9. The system of claim 8, wherein the aluminum tubing is disposed inside a stainless-steel sleeve.

10. A hydrogen peroxide vaporization system comprising:
a first process path for decontamination and comprising a boiler subsystem, the boiler subsystem configured to heat and vaporize a solution comprising hydrogen peroxide;
a second process path for conditioning and aerating a space comprising hydrogen peroxide and optionally comprising:
a desiccant accessory,
an air heater, and
a catalyst; and
one or more air moving devices configured for moving air about the solution comprising hydrogen peroxide.

11. The system of claim 10, wherein the boiler subsystem comprises:
a double helical flow path configured to heat the hydrogen peroxide solution from ambient temperature; and
a heat transfer contact area sized for continuous flow rates in a range of from about 0.5 to about 3 grams per minute.

12. The system of claim 11, wherein the heat transfer contact area is sized for continuous flow rates in a range of from about 1 to about 2 grams per minute.

13. The system of claim 10, wherein the one or more air moving devices comprise axial blowers.

14. The system of claim 10, wherein:
the solution comprising hydrogen peroxide comprises about 30 wt% to about 70% hydrogen peroxide; and
the boiler subsystem produces non-condensing vapor.

15. The system of claim 10, wherein the system is configured to decontaminate solutions having a volume in a no larger than 5 cubic meters.

16. The system of claim 10, wherein the boiler subsystem comprises a flexible aluminum tubing wrapped in a helical geometry around a cartridge heater.

17. The system of claim 10, wherein the boiler subsystem is fabricated using metal laser sintering.

18. A boiler for vaporizing hydrogen peroxide comprising:
a double helical flow path adapted to heat a hydrogen peroxide solution from ambient temperature;
a heat transfer contact area sized for continuous flow rates in a range of from about 0.5 to about 3 grams per minute;
a metal housing enclosing the double helical flow path; and
wherein the boiler is configured to produce non-condensing vapor from a solution comprising about 50 wt% to about 70% hydrogen peroxide.

19. The boiler of claim 18, wherein the double helical flow path comprises thin-walled aluminum tubing wrapped around a cartridge heater.

20. The boiler of claim 18, wherein the metal housing comprises a stainless-steel sleeve in contact with the wrapped tubing.
